(19) United States
(12) Patent Application Publication
(10) Pub. No.: US 2025/0041170 A1
(43) Pub. Date: Feb. 6, 2025

(54) OIL-BASED SOLID COMPOSITION FOR EXTERNAL USE

(71) Applicants: THE MENTHOLATUM COMPANY, Orchard Park, NY (US); ROHTO PHARMACEUTICAL CO., LTD., Osaka (JP)

(72) Inventor: Yoshiaki KAWAGUCHI, Orchard Park, NY (US)

(21) Appl. No.: 18/709,382

(22) PCT Filed: Nov. 22, 2022
(86) PCT No.: PCT/JP2022/043126

§ 371 Date: May 10, 2024

Related U.S. Application Data
(60) Provisional application No. 63/264,376, filed on Nov. 22, 2021.

(51) Int. Cl. A61K 8/02; A61K 8/34; A61K 8/63; A61K 8/81; A61K 8/92; A61Q 19/00

(52) U.S. Cl. CPC A61K 8/0229; A61K 8/345; A61K 8/63; A61K 8/8147; A61K 8/92; A61K 8/925; A61Q 19/001

(57) ABSTRACT

To provide an oil-based solid composition for external use having excellent stability. An oil-based solid composition for external use is prepared that comprises: (A) an oily component with a melting point of 55° C. or higher; (B) a polyhydric alcohol; and (C) an acrylic acid-based water-soluble polymer.

### TECHNICAL FIELD

The present invention relates to an oil-based solid composition for external use. More specifically, the present invention relates to an oil-based solid composition for external use comprising: (A) an oily component with a melting point of 55° C. or higher; (B) a polyhydric alcohol; and (C) an acrylic acid-based water-soluble polymer.

### BACKGROUND ART

Solid oil-based formulations are sometimes molded and used in stick form as preparations to be applied to the skin or lips, such as lip balm. Formulations molded in this way have the advantages of being easy to use and carry without contaminating the hands, and being able to be applied with precision to small areas. Formulations have been devised of solid oil-based compositions for external use that can be smoothly applied to the skin or mucous membranes (Patent Document 1).

These formulations, which use a solid oil-based agent at room temperature as the base, may be pre-dissolved in liquid phase and dispersed when an active component is to be added.

In general, however, oil-based formulations have a narrow selection of components that can be used, and it can be difficult to control their properties.

### PRIOR ART DOCUMENTS

### Patent Documents

[Patent Document 1] WO2019/189756 A1

### SUMMARY OF THE INVENTION

### Problem to Be Solved by the Invention

Although a polyhydric alcohol can be used as a dispersant for active components in the preparation of oil-based formulations, it has been found that the resulting solid oil-based formulation may separate.

In light of this situation, it is an object of the present invention to provide an oil-based solid composition for external use which has excellent stability.

### Means for Solving the Problem

The present inventors conducted extensive research on developing a stable formulation that does not experience separation when a polyhydric alcohol is used as a dispersant. As a result, they discovered that separation could be suppressed when an acrylic acid-based water-soluble polymer is added to an oil-based solid composition for external use. The present invention is a product of this discovery.

Specifically, the present invention relates to the following composition or method.
[1]

An oil-based solid composition for external use comprising:
(A) an oily component with a melting point of 55° C. or higher;
(B) a polyhydric alcohol; and
(C) an acrylic acid-based water-soluble polymer.
[2]

The oil-based solid composition for external use according to [1], wherein the composition is housed in a container with a cylindrical, polygonal, spherical, domed, hemispherical, conical, pyramidal, conical trapezoidal, or pyramidal trapezoidal shape.
[3]

The oil-based solid composition for external use according to [1] or [2], further comprising (D) a steroid.
[4]

The oil-based solid composition for external use according to any one of [1] to [3], wherein (A) the oily component with a melting point of 55° C. or higher is one component or a combination of two or more components selected from the group consisting of paraffin wax, microcrystalline wax, ceresin, polyethylene, and beeswax.
[5]

The oil-based solid composition for external use according to any one of [1] to [4], wherein the (B) polyhydric alcohol includes dipropylene glycol.
[6]

The oil-based solid composition for external use according to any one of [1] to [5], wherein the (C) acrylic acid-based water-soluble polymer is one component or two or more components selected from the group consisting of polyacrylic acid, cross-linked polyacrylic acid, and alkyl acryl copolymers.
[7]

A method for increasing the stability of a composition comprising (A) an oily component with a melting point of 55° C. or higher and (B) a polyhydric alcohol, by the additional presence of (C) an acrylic acid-based water-soluble polymer in the composition.

### Effect of the Invention

The present invention is able to provide an oil-based solid composition for external use which has excellent stability.

### MODE FOR CARRYING OUT THE INVENTION

### Definitions

In the present specification, “solid composition” refers to a composition that has a hardness of 5 (g) or greater at 25° C. when the product is kept in an isothermal state by cooling at approximately 25° C. for at least 12 hours after filling the container. The hardness is preferably 10 to 100 (g), more preferably 15 to 80 (g), even more preferably 15 to 65 (g), and still more preferably 20 to 50 (g). The measurement of hardness in the present invention is performed on formulations that have been kept in an isothermal state at approximately 25° C. for at least 12 hours after a lip container has been filled with the composition. Specifically, this means the maximum value for hardness (unit: g) obtained by a rheometer (Sun Rheometer CR-100 from Sun Scientific Corporation) before a cylindrical adapter with a diameter of 1 mm has entered the formulation at a rate of 20 mm/min to 10 mm. The inner dish (bucket) of the lipstick container is made of polypropylene, and the body (base) is made of ABS resin.

In the present specification, “melting point” refers to what is obtained using the third “melting point measurement method” described in the “Standards for Cosmetic Components.” In other words, the device uses a beaker filled with water as the bath and heating container. A thermometer with an immersion wire or a fully submerged thermometer is used. The sample is gradually heated to 90 to 92° C. while stirring thoroughly until melted. Heating is then stopped and the sample is allowed to cool until the temperature is 8 to 10° C. above the melting point of the sample. The thermometer is cooled to around 5° C. and wiped dry, and then half of the mercury sphere is immediately inserted into the sample, immediately removed, placed vertically, and allowed to cool. When the adhering sample becomes cloudy, it is immersed in water below 16° C. for 5 minutes. The thermometer is then inserted into the test tube, and a cork stopper is used to secure the thermometer so that the distance between the bottom of the thermometer and the bottom of the test tube is 15 mm. The test tube is suspended in a beaker of water at about 16° C. and heated at a rate of 2° C. per minute until the temperature of the bath reaches 30° C., at which time it is heated at a rate of 1° C. per minute. The temperature is measured when the first drop of the melted sample leaves the thermometer. This operation is performed three times. If the difference between the measured values is less than 1° C., the average value is used as the melting point. If the difference is greater than 1° C., the operation is repeated two more times, and the average value from a total of five tests is used as the melting point.

### Oil-Based Solid Composition for External Use

The present invention relates to an oil-based solid composition for external use comprising:
(A) an oily component with a melting point of 55° C. or higher;
(B) a polyhydric alcohol; and
(C) an acrylic acid-based water-soluble polymer.

### (A) Oily component with Melting Point of 55° C. or Higher

The oily components with a melting point of 55° C. or higher used in the present invention can be any grade of oil used as components in topical products in the fields of pharmaceuticals and cosmetics, and are not limited as long as the melting point is 55° C. or higher. Examples of oily components with a melting point of 55° C. or higher include hydrocarbons such as paraffin wax, isoparaffin wax, polyethylene, microcrystalline wax, ceresin, and ozokerite; ester oils such as beeswax, whale wax, synthetic beeswax, white beeswax, Japan wax, synthetic Japan wax, carnauba wax, candelilla wax, rice bran wax, sunflower wax, hydrogenated jojoba oil, hardened castor oil, hydrogenated palm oil, hardened palm oil, and glyceryl behenate eicosanedioic acid; higher alcohols such as stearyl alcohol, arachyl alcohol, behenyl alcohol, and carnabyl alcohol; cholesterol; and higher fatty acids such as palmitic acid, stearic acid, behenic acid, and 12-hydroxystearic acid. Oily components with a melting point of 55° C. or higher may be used alone or in combinations of two or more. The oily component with a melting point of 55° C. or higher used in the present invention preferably includes one, or a combination of two or more, of the following: paraffin wax, microcrystalline wax, ceresin, polyethylene, and beeswax.

The total amount of component (A) is preferably 1% by mass or more, more preferably 2% by mass or more, even more preferably 3% by mass or more, still more preferably 4% by mass or more, yet more preferably 6% by mass or more, and most preferably 8% by mass or more relative to the mass of the oil-based solid composition for external use from the standpoint of realizing the effect of the invention to a greater extent. The total amount of component (A) is preferably 50% by mass or less, more preferably 47.5% by mass or less, even more preferably 45% by mass or less, still more preferably 42.5% by mass or less, yet more preferably 40% by mass or less, and most preferably 35% by mass or less relative to the mass of the oil-based solid composition for external use from the standpoint of realizing the effect of the invention to a greater extent. The total amount of (A) component is preferably 1% by mass to 50% by mass, more preferably 2% by mass to 47.5% by mass, even more preferably 3% by mass to 45% by mass, still more preferably 4% by mass to 42.5%, yet more preferably 6% by mass to 40% by mass, and most preferably 8% by mass to 35% by mass relative to the mass of the oil-based solid composition for external use from the standpoint of realizing the effect of the invention to a greater extent.

### (B) Polyhydric Alcohol

The polyhydric alcohols used in the present invention are not limited and may be any grade that is liquid at 25° C. and used as a component in topical products in the fields of pharmaceuticals and cosmetics. Polyhydric alcohols include glycerin, diglycerin, triglycerin, propylene glycol, dipropylene glycol, 1,4-butanediol, ethylene glycol, diethylene glycol, polyethylene glycol (liquid at room temperature), isoprene glycol, pentylene glycol, and 1,3-butylene glycol. These polyhydric alcohols may be used alone or in combinations of two or more. The polyhydric alcohols used in the present invention preferably include, at least, dipropylene glycol.

The overall amount of component (B) is preferably 0.1% or more by mass, more preferably 0.5% or more by mass, more preferably 1% or more by mass, and especially preferably 3% or more by mass relative to the mass of the oil-based solid composition for external use from the standpoint of realizing the effect of the invention to a greater extent. The overall amount of component (B) is preferably 40% by mass or less, more preferably 37% by mass or less, even more preferably 34% by mass or less, and still more preferably 30% by mass or less relative to the mass of the oil-based solid composition for external use from the standpoint of the feel of the formulation and ease of handling. The overall amount of component (B) is preferably 0.1 to 40% by mass, more preferably 0.5 to 37% by mass, even more preferably 1 to 34% by mass, still more preferably 3 to 31% by mass, yet more preferably 15 to 30% by mass, and most preferably 23 to 30% by mass relative to the mass of the oil-based solid composition for external use from the standpoint of realizing the effect of the invention to a greater extent.

In the oil-based solid composition for external use of the present invention, from the standpoint of realizing the effect of the invention to a greater extent, the ratio of the amount of component (B) to component (A) can be, for example, 0.5 to 3 parts by mass, preferably 0.8 to 2 parts by mass, and more preferably 1 to 1.5 parts by mass of component (B) per 1 part by mass of component (A).

### (C) Acrylic Acid-Based Water-Soluble Polymer

The acrylic acid-based water-soluble polymer (C) used in the present invention is not particularly limited, and may be any grade used as a component in topical agents in the fields of pharmaceuticals and cosmetics. Examples of acrylic acid-based water-soluble polymers include polyacrylic acid, cross-linked polyacrylic acid (carboxyvinyl polymer), and alkyl acryl copolymers. Alkyl acrylate copolymers include, for example, (acrylates/alkyl acrylate (C10-30)) crosspolymers.

The acrylic acid-based water-soluble polymer can also be used as a pharmaceutically acceptable salt. Examples include salts with organic bases (for example, salts with tertiary amines such as trimethylamine, triethylamine, monoethanolamine, triethanolamine, pyridine salts, and basic ammonium salts such as arginine and glycine), and salts with inorganic bases (for example, sodium salts, potassium salts and other alkali metal salts, calcium salts, magnesium salts and other alkaline earth metal salts, aluminum salts, and ammonium salts, etc.). Especially preferred salts include sodium, potassium, triethanolamine, and arginine salts.

These acrylic acid-based water-soluble polymers may be used alone or in combinations of two or more. The acrylic acid-based water-soluble polymer used in the present invention is preferably one or a combination of two or more polymers selected from the group consisting of polyacrylic acid, alkyl acryl copolymers, and cross-linked polyacrylic acid.

The amount of component (C) is preferably 0.1% by mass or more, more preferably 0.2% by mass or more, even more preferably 0.3% by mass or more, and still more preferably 0.5% by mass or more relative to the mass of the oil-based solid composition for external use from the standpoint of realizing the effect of the invention to a greater extent. The amount of component (C) is preferably less than 5% by mass, more preferably less than 4% by mass, even more preferably less than 3% by mass, and still more preferably less than 2% by mass relative to the mass of the oil-based solid composition for external use from the standpoint of realizing the effect of the invention to a greater extent. The amount of (C) is preferably 0.1 to 5% by mass, more preferably 0.2 to 4% by mass, even more preferably 0.3 to 3% by mass, and still more preferably 0.5 to 2% by mass relative to the mass of the oil-based solid composition for external use. While not limited, it is desirable to include 1.0 to 1.5% by mass of carboxyvinyl polymer (for example, Carbopol 980).

The ratio of component (C) to component (A) can be, for example, 0.001 to 0.1 parts by mass, preferably 0.003 to 0.05 parts by mass, and more preferably 0.015 to 0.04 parts by mass of component (C) per 1 part by mass of component (A).

### (D) Steroid

In addition to components (A), (B), and (C), the oil-based solid composition for external use in the present invention may optionally contain a steroid (D). The steroid (D) in the present invention is not particularly limited and may be any grade used as a component in topical agents in the field of pharmaceuticals.

Steroids (D) include, for example, prednisolone, hydrocortisone, cortisone, dexamethasone, and derivatives or salts thereof.

Derivatives typically include esters (especially esters with organic or inorganic acids), salts, and ester salts. The steroid can also be a hydrate, hemihydrate, or anhydride.

Esters include, for example, esters of organic acids such as valeric acid, acetic acid, succinic acid, butyric acid (butanoic acid), propionic acid, sulfobenzoic acid, caprylic acid, palmitic acid, furan carboxylic acid, pivalic acid; and esters of inorganic acids such as phosphoric acid. Each molecule may also contain more than one ester. When more than one ester is contained in a molecule, it may be more than one ester of a single acid, or esters of two or more acids.

Salts include salts with organic bases (such as methylamine salts, triethylamine salts, triethanolamine salts, morpholine salts, piperazine salts, pyrrolidine salts, tripyridine salts, picoline salts and other organic amine salts), and salts with inorganic bases (ammonium salts, alkaline metal salts such as sodium and potassium salts, alkaline earth metal salts such as calcium and magnesium salts, and metal salts such as zinc and aluminum salts). Alkali metal salts such as sodium and potassium salts are especially preferred.

Specific examples of derivatives of compounds mentioned above include prednisolone esters such as prednisolone valerate acetate (PVA), prednisolone succinate, prednisolone acetate and prednisolone phosphate, dexamethasone esters such as dexamethasone valerate, dexamethasone propionate, dexamethasone acetate, dexamethasone phosphate, dexamethasone metasulfobenzoate, dexamethasone caprylate and dexamethasone palmitate, and hydrocortisone esters such as hydrocortisone butyrate (especially hydrocortisone-17-butyrate), hydrocortisone acetate, hydrocortisone succinate, hydrocortisone butyrate, hydrocortisone butyrate propionate, and hydrocortisone phosphate.

One or more steroids selected from the group consisting of prednisolone valerate acetate, dexamethasone acetate, hydrocortisone, and hydrocortisone acetate is preferred, with hydrocortisone being especially preferred.

The amount of component (D) is preferably 0.01 to 3% by mass, more preferably 0.03 to 2.5% by mass, even more preferably 0.05 to 2% by mass, and still more preferably 0.1 to 1% by mass relative to the mass of the oil-based solid composition for external use from the standpoint of realizing the effect of the invention to a greater extent.

The ratio of component (D) to component (B) can be, for example, 0.005 to 0.1 parts by mass, more preferably 0.008 to 0.06 parts by mass, even more preferably 0.01 to 0.05 parts by mass, and still more preferably 0.02 to 0.04 parts by mass of component (D) per 1 part by mass of component (B) in the oil-based solid composition for external use from the standpoint of realizing the effect of the invention to a greater extent.

### Water

The oil-based solid composition for external use according to the present invention may optionally contain water in addition to components (A), (B), and (C), but preferably the composition is substantially water-free. The amount of water relative to the total mass of the oil-based solid composition for external use according to the present invention is preferably less than 5% by mass, more preferably less than 1% by mass, and even more preferably less than 0.1% by mass.

### Base, Carrier, or Other Component

The oil-based solid compositions for external use according to the present invention may contain substrates or carriers commonly used in pharmaceuticals or cosmetics to the extent that the effect of the present invention is not impaired. The oil-based solid compositions for external use according to the present invention may also contain additives such as oily components with a melting point below 55° C., surfactants, thickeners other than component (C), humectants, preservatives, antioxidants, clarifying agents, preservatives, coloring agents, fragrances, and dyes. One or more of these additives can be used alone or in combinations of two or more.

Oily components with a melting point below 55° C. are not particularly limited and can be any grade of oil used as a component in topical products in the fields of pharmaceuticals and cosmetics as long as the melting point is less than 55° C. Oily components with a melting point below 55° C. include, for example, hydrocarbons, ester oils, higher alcohols, higher fatty acids, and silicone oils with melting points below 55° C.

Hydrocarbons with melting points below 55° C. include petroleum jelly, mineral oil, hydrogenated polydecene, polybutene, hydrogenated polyisobutene, alpha-olefin oligomers, liquid isoparaffin, liquid paraffin, and squalane. Among these, petroleum jelly, and squalane are especially preferred.

Ester oils with a melting point below 55° C. include glyceryl trioctanoate, avocado oil, olive oil, sesame oil, rice bran oil, safflower oil, soybean oil, corn oil, rape seed oil, castor oil, cottonseed oil, mink oil, isopropyl myristate, butyl myristate, isopropyl palmitate, ethyl oleate, ethyl linoleate, isopropyl linoleate, cetyl caprylate, hexyl laurate, decyl myristate, decyl oleate, oleyl oleate, isostearyl laurate, isotridecyl myristate, isocetyl myristate, isostearyl myristate, octyldodecyl myristate, octyl palmitate, cetyl palmitate, isocetyl palmitate, isostearyl palmitate, diethyl sebacate, diisopropyl adipate, isopropyl palmitate, propylene glycol dioleate, isodecyl oleate, isopropyl isostearate, cetyl 2-ethylhexanoate, stearyl 2-ethylhexanoate, propylene glycol dicaprate, propylene glycol dioleate, glyceryl tri-2-ethylhexanoate, glyceryl tri(caprylic/capric acid), isononyl isononanoate, diisopropyl sebacate, propylene glycol isostearate, triethylhexanoin, triisostearate, diisostearyl malate, triisostearyl citrate, di(phytosteryl/octyldodecyl) lauroyl glutamate, fatty acid (C10-30) (cholesterol/lanosterol) esters, lanolin, shea butter, jojoba oil, coconut oil, dimer dilinoleic acid (phytosteryl/isostearyl/cetyl/stearyl/behenyl), dimer dilinoleic acid, tri (caprylic/capric/myristic/stearic acid) glyceryl, phytosteryl macadamia nut oil fatty acid, hexa (hydroxystearate/stearate/rosinic acid) dipentaerythristyl, pentaerythristyl tetraethylhexanoate, and tripolyhydroxystearate. Among these, tri(caprylic/capric) glyceryl, jojoba oil, and dipentaerythryl tripolyhydroxystearate are especially preferred.

Higher alcohols with melting points below 55° C. include, for example, lauryl alcohol, octyldodecanol, myristyl alcohol, cetanol, and oleyl alcohol. Among these, cetanol is preferred.

Higher fatty acids with melting points below 55° C. include oleic acid, isostearic acid, capric acid, lauric acid, and myristic acid.

The amount of an oily component with a melting point below 55° C. is preferably 34.5 to 60% by mass, 38.5 to 55% by mass and 40 to 47% by mass relative to the total mass of oil-based solid composition for external use.

The surfactant can be a nonionic, cationic, anionic, or amphoteric surfactant. Examples of nonionic surfactants include sorbitan fatty acid esters such as sorbitan monoisostearate, sorbitan monolaurate, sorbitan monopalmitate, sorbitan monostearate, diglycerol sorbitan penta-2-ethylhexylate, and diglycerol sorbitan tetra-2-ethylhexylate; propylene glycol fatty acid esters such as propylene glycol monostearate; hardened castor oil derivatives such as polyoxyethylene hardened castor oil 40 (HCO-40), polyoxyethylene hardened castor oil 50 (HCO-50), polyoxyethylene hardened castor oil 60 (HCO-60), and polyoxyethylene hardened castor oil 80; polyoxyethylene sorbitan fatty acid esters such as polyoxyethylene (20) sorbitan monolaurylate (polysorbate 20), polyoxyethylene (20) sorbitan monostearate (polysorbate 60), polyoxyethylene (20) sorbitan monooleate (polysorbate 80), and polyoxyethylene (20) sorbitan isostearate; polyoxyethylene glyceryl mono coconut oil fatty acid; glycerol alkyl ethers; glycerol fatty acid esters such as glyceryl stearate, glyceryl myristate, glyceryl oleate, and glyceryl isostearate; polyglycerol fatty acid esters such as polyglyceryl-2 oleate, polyglyceryl-2 stearate, polyglyceryl-10 oleate, polyglyceryl-10 stearate, polyglyceryl-10 laurate, polyglyceryl-10 distearate, polyglyceryl-10 trioleate, polyglyceryl-10 pentaoleate polyglyceryl-10 pentastearate, and polyglyceryl-6 polyricinoleate; alkylglucosides; polyoxyethylene alkyl ethers such as polyoxyethylene stearyl ether, polyoxyethylene lauryl ether, polyoxyethylene cetyl ether, and polyoxyethylene behenyl ether; polyethylene glycol fatty acid esters such as polyethylene glycol monostearate and PEG-30 dipolyhydroxystearate; and sucrose fatty acid esters such as sucrose hexaisostearate. Among these, polyglyceryl-10 oleate and PEG-30 dipolyhydroxystearate are especially preferred.

Thickening agents other include than component (C) (vinylpyrrolidone/eicosene) copolymers, (polyglyceryl-2 isostearate/dimarjolinoleic acid) copolymers, hydrogenated castor oil with dimer dilinoleic acid, myristic acid dextrin, and palmitic acid dextrin.

Examples of humectants include hyaluronic acid and salts or derivatives thereof (for example, sodium hyaluronate, zinc hyaluronate, low molecular weight hyaluronic acid, acetylated hyaluronic acid, cross-linked hyaluronic acid derivatives, cationized hyaluronic acid, etc.); chondroitin sulfate and salts thereof; heparin analogues; amino acids such as alanine, serine, aspartic acid, glycine, arginine and derivatives thereof; alkylene oxides such as PPG-17 buteth-17, PPG-25 sorbitol, polyoxyalkylene alkyl and glucosides, PEG/PPG/polybutylene glycol-8/5/3 glycerin, polyoxyalkylene glyceryl; glycosyl trehalose and trehalose; ceramides; phospholipids; MPC polymers; NMF-derived components such as lactic acid, sodium lactate, sodium pyrrolidonecarboxylate, and urea; collagen, elastin, keratin, chitin, chitosan, and hydrolysates thereof; hydroxyethylurea; honey; and plant-derived components (for example, aloe, seaweed, cassia, chlorella, lemongrass, chamomile, hamamelis, chia, shiso, grapefruit, amachazuru, etc.).

Preservatives include, for example, benzoic acid, acetic acid, phenol, iodine tincture, paraoxybenzoic esters, chlorobutanol, chlorocresol, benzyl alcohol, phenethyl alcohol, and dehydroacetic acid.

Antioxidants include, for example, sulfites and ascorbic acid.

Antioxidants include, for example, dibutylhydroxytoluene (BHT), butylhydroxyanisole (BHA), sodium sulfite, erythorbic acid, L-cysteine hydrochloride, vitamin C, and vitamin E. Vitamin C includes, for example, ascorbigen-A, ascorbic acid stearate, ascorbic acid palmitate, L-ascorbyl dipalmitate, ascorbic acid, sodium ascorbate, dehydroascorbic acid, sodium ascorbate phosphate, sodium ascorbate phosphate salt, and magnesium ascorbate phosphate ester. Vitamin E includes, for example, dl-alpha-tocopherol, dl-alpha-tocopherol acetate, dl-alpha-tocopherol succinate, and dl-alpha-tocopheryl calcium succinate.

Cooling agents include, for example, menthol (I-menthol, dl-menthol, etc.), camphor (d-camphor, dl-camphor, etc.), terpenoids such as borneol, essential oils containing terpenoids (such as peppermint oil), and pharmacologically acceptable salts of these. These agents may be used alone or in combinations of two or more as appropriate.

Preservatives include, for example, benzoic acid, sodium benzoate, dehydroacetic acid, sodium dehydroacetate, isobutyl paraoxibenzoate, isopropyl paraoxibenzoate, butyl paraoxibenzoate, ethyl paraoxibenzoate, propyl paraoxibenzoate, benzyl paraoxibenzoate methyl p-hydroxybenzoate, and phenoxyethanol.

Colorants include, for example, inorganic pigments and natural dyes. Inorganic pigments include barium sulfate, aluminum hydroxide, synthetic phlogopite, mica, silica, tin oxide, titanium dioxide, iron oxide, and alumina.

The oil-based solid composition for external use according to the present invention may also contain other active ingredients to the extent that the effect of the invention is not impaired. Specific examples of these ingredients include local anesthetics, anti-inflammatory agents, bactericides, antihistamines, antipruritic components, blood circulation promoting components, and TRPV1 agonists.

Local anesthetics include lidocaine, dibucaine, and ethyl aminobenzoate.

Anti-inflammatory agents include, for example, allantoin and derivatives thereof (for example, alcloxa, allantoin, etc.), glycyrrhizic acid and derivatives or salts thereof (for example, dipotassium glycyrrhizate, monoammonium glycyrrhizate, etc.), glycyrrhetinic acid and derivatives or salts thereof (for example, glycyrrhetinic acid, stearyl glycyrrhetinate, etc.), zinc oxide, tocopherol acetate, menthol, camphor, and turpentine oil.

Bactericides include, for example, benzalkonium chloride, acrinol, ethanol, benzethonium chloride, cetyltrimethylammonium chloride, cresol, poppidone-iodine, potassium iodide, iodine, isopropyl methylphenol, parabens, phenoxyethanol cetylpyridinium chloride cetylpyridinium chloride, miconazole and salts thereof, chlorobutanol, and plant-derived ingredients (for example, aloe, clara, rosemary, mulberry, eucalyptus, quina, and clove).

Antihistamines include, for example, ethanolamine antihistamines such as diphenhydramine, bromodiphenhydramine and diphenylpyraline, propylamine antihistamines such as chlorpheniramine, phenothiazine antihistamines such as isothipendyl, and salts of these.

Examples of antipruritic agents include crotamiton.

Blood circulation promoting components include plant-derived components (for example, horse chestnut, Ashitaba, Arnica, Ginkgo biloba, Ukyou, plants of the genus Plectranthus, Dutch oak, chamomile, Roman chamomile, Gentian, burdock, rice, hawthorn, shitake mushroom, ginger, Rehmannia root, Japanese apricot, Japanese parsley, Japanese harlequin, Chinese parsley, thyme, Chinese juniper, spruce, spruce nigrum, carrot, ginseng, garlic, butcherbroom, grape, button, horse chestnut, melissa, yuzu, yokuinin, rosemary, rose hips, peach, apricot, walnut, and maize); as well as caffeine, Capsicum tincture, acetylcholine, ictamol, cantharis tincture, gamma oryzanol, capsaicin, shoukou tincture, zingerone, cephalanthin, dl-alpha-tocopherol acetate, tocopherol nicotinate, nicotinic acid benzyl esters, glucosyl hesperidin, and hesperidin.

TRPV1 agonists include, for example, capsaicin.

### Characteristics

The oil-based solid composition for external use in the present invention is a solid composition at 25° C. or lower, preferably a molded composition. Here, “molded” means the product is not fluid and maintains a constant shape at a temperature of 25° C. or lower when no external force is applied.

The oil-based solid composition for external use in the present invention is preferably a composition molded into a cylindrical, polygonal, spherical, dome, hemispherical, conical, pyramidal, conical trapezoidal, or pyramidal trapezoidal shape. Cylindrical or polygonal shapes, also called stick shapes, are shapes whose axial length is longer and circumferential diameter is shorter. The circumferential cross-sectional shape is not limited, and can be a circle, oval, square, or irregular shape. The axial end surfaces (top and/or bottom end surfaces) may be flat, convex, or concave. Although not limited, the mass per unit (per formulation) should be 2 to 20 g, but 4 to 15 g is more preferred. The formulation can also be made with a unit mass that is greater than 20 g.

### Applications

The oil-based solid composition for external use in the present invention is preferably used for, but not limited to, application to various skin diseases and conditions, such as erythema, eczema, itching, rash, inflammation, athlete's foot, and keratosis. It can also be conveniently applied to scalp eczema (specifically, seborrheic eczema of the scalp), which often requires quick treatment and improvement when symptoms such as itching arise.

### Application Sites

The oil-based solid composition for external use in the present invention can be applied to the skin or mucous membranes, and is especially preferred as a topical skin composition. This includes skin on the hands (palms and fingers), face, thighs, shins, feet, head, neck, chest, armpits, back, waist, arms, back of elbows, and back of knees.

The way in which an oil-based solid composition for external use in the present invention is used varies depending on the condition of the skin, stratum corneum, etc., and the age, gender, etc. of the user. For example, the following method may be used. An appropriate amount (for example, about 0.5 to 2 g) may be applied several times a day (for example, about 1 to 5 times a day, preferably 1 to 3 times a day, and more preferably once a day).

### Container

There are no particular restrictions on the container housing the oil-based solid composition for external use in the present invention. The external shape of the container is preferably cylindrical, polygonal, spherical, dome-shaped, hemispherical, conical, pyramidal, conical trapezoidal, or pyramidal. The protruding portion of the oil-based solid composition for external use protruding from the body of the container may be molded by the interior space of the lid. While not limited to this example, a container with a diameter of 1 to 4 cm and a height of 6 to 10 cm can be used. For example, the oil-based solid composition for external use can be housed in a cylindrical container that is 1.6 cm in diameter and 6.7 cm in height, that is 1.5 cm in diameter and 8.2 cm in height, or that is about 3.3 cm in diameter and 9.1 cm in height.

When the container body for the oil-based solid composition for external use in the present invention has a mechanism that enables the oil-based solid composition for external use to protrude from the container body, the protruding portion of the oil-based solid composition for external use preferably has a protruding dimension of 10 to 100 mm, more preferably 20 to 80 mm.

As for the container material, some or all, preferably all, of the contact surface of the container with the oil-based solid composition for external use is composed of at least one material selected from the group consisting of polyolefin resin, acrylic acid resin, polyester, polycarbonate, fluororesin, polyvinyl chloride, polyamide, ABS resin, AS resin, polyacetal, modified polyphenylene ether, polyarylate, polysulfone, polyimide, cellulose acetate, aluminum, and glass.

### Production Method

The oil-based solid composition for external use in the present invention can be produced using any method common in the art. Preferred examples include: dispersing the acrylic acid-based water-soluble polymer in a polyhydric alcohol and then supplying the dispersion to the oily component; mixing in the polyhydric alcohol after first dispersing the acrylic acid-based water-soluble polymer in the oily component; and preparing a pre-dispersed phase containing a high concentration of the acrylic acid-based water-soluble polymer and an oily component that is liquid oil at room temperature and then mixing this phase with the oil phase. A sterilization step or filtration step, for example, can be included in these methods, if necessary.

### Stabilizer

The present invention relates to a stabilizer for a composition containing (A) an oily component with a melting point of 55° C. or higher, (B) a polyhydric alcohol, and (C) an acrylic acid-based water-soluble polymer. Stabilization here includes, but is not limited to, suppressing separation of the composition. The same conditions as those for the concentration, hardness, and formulation of each component in the oil-based solid composition for external use of the present invention described above can be applied to the stabilizer.

### Composition Stabilizing Method

The present invention also relates to a method for increasing the stability of a composition comprising (A) an oily component with a melting point of 55° C. or higher and (B) a polyhydric alcohol, by the additional presence of (C) an acrylic acid-based water-soluble polymer in the composition. The same conditions as those for the concentration, hardness, and formulation of each component in the oil-based solid composition for external use of the present invention described above can be applied to this method.

The present invention encompasses the following aspects.
[1]

An oil-based solid composition for external use comprising:
(A) an oily component with a melting point of 55° C. or higher that is a combination of one or more type selected from the group consisting of paraffin wax, microcrystalline wax, ceresin, polyethylene, and beeswax;
(B) a polyhydric alcohol; and
(C) one or more acrylic acid-based water-soluble polymers selected from the group consisting of polyacrylic acid, cross-linked polyacrylic acid, and/or alkyl acryl copolymers.
[2]

The oil-based solid composition for external use according to [1] above, wherein the oil-based solid composition for external use is housed in a container with a cylindrical, polygonal, spherical, domed, hemispherical, conical, pyramidal, conical trapezoidal, or pyramidal trapezoidal shape.
[3]

The oil-based solid composition for external use according to [1] or [2] above, further comprising a steroid (D).
[4]

The oil-based solid composition for external use according to any one of [1] to [3] above, wherein the steroid (D) is one or more type selected from the group consisting of prednisolone valerate acetate, dexamethasone acetate, hydrocortisone, and hydrocortisone acetate.
[5]

The oil-based solid composition for external use according to any one of [1] to [4] above, wherein the steroid (D) is hydrocortisone.
[6]

The oil-based solid composition for external use according to any one of [1] to [5] above, wherein the polyhydric alcohol (B) is selected from the group consisting of glycerin, diglycerin, triglycerin, propylene glycol, dipropylene glycol, 1,4-butanediol, ethylene glycol, diethylene glycol, polyethylene glycol (liquid at room temperature), isoprene glycol, pentylene glycol, and 1,3-butylene glycol.
[7]

The oil-based solid composition for external use according to any one of [1] to [6] above, wherein the polyhydric alcohol (B) includes dipropylene glycol.
[8]

The oil-based solid composition for external use according to any one of [1] to [7] above, wherein the acrylic acid-based water-soluble polymer (C) is polyacrylic acid.
[9]

The oil-based solid composition for external use according to any one of [1] to [8] above, wherein the amount is 0.1% or less by mass relative to the overall mass of the oil-based solid composition for external use.
[10]

The oil-based solid composition for external use according to any one of [1] to [9] above, wherein the amount of (A) is 8% to 35% by mass relative to the overall mass of the oil-based solid composition for external use.
[11]

The oil-based solid composition for external use according to any one of [1] to [9] above, wherein the amount of (B) is 15% to 30% by mass relative to the overall mass of the oil-based solid composition for external use.
[12]

The oil-based solid composition for external use according to any one of [1] to [11] above, wherein the ratio of the amount of component (B) to component (A) is 0.5 to 3 parts by mass of component (B) per 1 part by mass of component (A).
[13]

The oil-based solid composition for external use according to any one of [1] to [12] above, wherein the amount of (C) is 0.5% to 2% by mass relative to the overall mass of the oil-based solid composition for external use.
[14]

The oil-based solid composition for external use according to any one of [1] to [13] above, wherein the ratio of the amount of component (C) to component (A) is 0.001 to 0.1 parts by mass of component (C) per 1 part by mass of component (A).
[15]

The oil-based solid composition for external use according to any one of [3] to [5] above, wherein the ratio of the amount of component (D) to component (B) is 0.005 to 0.1 parts by mass of component (D) per 1 part by mass of component (B).
[16]

The oil-based solid composition for external use according to any one of [1] to [15] above, further comprising an oily component with a melting point lower than 55° C.
[17]

The oil-based solid composition for external use according to any one of [1] to [16] above, wherein the amount of the oily component with a melting lower than 55° C. is 34.5 to 60% by mass relative to the overall mass of the oil-based solid composition for external use.

### EXAMPLES

The present invention will now be described in greater detail with reference to examples. The present invention is not limited to the examples described below.

All values shown in the tables below are mass percentages unless otherwise specified. All homogenizers used were T.K. Robomics and Homomixer Mark II Model 2.5 from Tokushu Kika Kogyo Co., Ltd.

### Example 1

The composition of Example 1 was prepared as shown in Table 1 and used to fill a container. Specifically, dipropylene glycol was mixed with carboxyvinyl polymer (Aqupec 505E; Sumitomo Seika Co., Ltd.) to prepare the polyhydric alcohol phase, and the mixture was stirred at 90° C. The oil phase components were prepared separately by mixing at 90° C. The resulting polyhydric alcohol phase was combined with the oil phase and processed in a homogenizer at 8,000 rpm for 1 minute under 90° C. heat. The resulting emulsion was used to fill a lip container, which was then allowed to stand while refrigerated at 4° C. for 3 hours. Here, the inner dish (bucket) of the lipstick container was made of polypropylene, and the body (base) was made of ABS resin.

### Examples 2 and 3

The compositions of Examples 2 and 3 were prepared as shown in Table 1 and used to fill containers. Specifically, the oil phase components was prepared by mixing at 90° C., and then carboxyvinyl polymer (Aqupec 505E; Sumitomo Seika Co., Ltd.) was added and mixed in. The oil phase containing the carboxy vinyl polymer was mixed with the polyhydric alcohol phase and processed in a homogenizer at 8,000 rpm for 1 minute under 90° C. heat. The resulting emulsion was placed in a container as in Example 1 and allowed to stand while refrigerated at 4° C. for 3 hours.

### Examples 4 and 5

The compositions of Examples 4 and 5 were prepared as shown in Table 1 and used to fill containers. Specifically, a pre-dispersed phase consisting of squalane and carboxyvinyl polymer (Aqupec 505E; Sumitomo Seika Co., Ltd.). The pre-dispersed phase was supplied to a mixture of oil phase components prepared separately at 90° C. and stirred. The oil phase including the pre-dispersed phase was mixed with dipropylene glycol and processed in a homogenizer at 8,000 rpm for 1 minute under 90° C. heat. The resulting emulsion was used to fill a container as in Example 1 and allowed to stand while refrigerated at 4° C. for 3 hours.

### Comparative Example 1

The composition was prepared in the same manner as in Example 1, but without a carboxy vinyl polymer, and used to fill a container as in Example 1, which was allowed to stand for 3 hours while refrigerated at 4° C.

### Test Example 1. Stabilization Test

The compositions of the examples shown in Table 1 below were stored on an indoor window sill from Sep. 1, 2020 to Mar. 1, 2021 at temperatures and humidities that would be expected.

The properties were visually checked under fluorescent light. The area occupied by the liquid substance (possibly dipropylene glycol) on the surface of the formulation due to separation was evaluated as a percentage of the total surface area of the formulation.
⊙: No separation
◯: Percent separated is more than 0% but less than 30%
Δ: Percent separated is equal or more than 30% but less than 80%
x: Percent separated is equal or more than 80%

### Table 1

The test results confirmed that separation was suppressed in the compositions of the examples.

### Examples 6 and 7

The compositions of Examples 6 and 7 were prepared as shown in Table 2 and used to fill containers. Specifically, the components of the oil phase were prepared by mixing at 90° C. Separately, a pre-dispersed phase of carboxyvinyl polymer (Carbopol 980; Lubrizol) in squalane was prepared at room temperature. The pre-dispersed phase was then supplied to the oil phase at 90° C. Next, dipropylene glycol heated to 90° C. was added, and mixing performed in a homogenizer at 7,000 rpm for 1 minute at 90° C. Afterward, the composition was used to fill a jar-type container (Plastic Jar No. A-2, 11 cc: M.I. Chemical Co., Ltd.) and allowed to stand at 4° C. for 1 hour.

### Examples 8 and 9

As in Examples 6 and 7, the compositions of Examples 8 and 9 were prepared with the components and amounts shown in Table 2 and used to fill containers. However, instead of dipropylene glycol heated to 90° C., a mixture of dipropylene glycol and hydrocortisone heated to 90° C. was used, and the mixture was supplied to the oil phase. The resulting emulsions were used to fill containers as in Examples 6 and 7 and allowed to stand at 4° C. for 1 hour. Carbopol 980 (Lubrizol) was used in Example 8 as the carboxyvinyl polymer, and Aqupec 505E (Sumitomo Seika Co., Ltd.) in Example 9.

### Example 10

As in Examples 6 and 7, the composition of Example 10 was prepared with the components and amounts shown in Table 2 and used to fill a container. However, instead of a carboxyvinyl polymer, an (acrylates-acrylic acid alkyl (C10-30) crosspolymer (Pemulen EZ4U) was used. The resulting emulsion was used to fill a container as in Examples 6 and 7 and allowed to stand at 4° C. for 1 hour.

### Example 11

As in Examples 8 and 9, the composition of Example 11 was prepared with the components and amounts shown in Table 2 and used to fill a container. However, instead of a carboxyvinyl polymer, an (acrylates-acrylic acid alkyl (C10-30) crosspolymer (Pemulen TR1; Lubrizol) was used. The resulting emulsion was used to fill a container as in Examples 8 and 9 and allowed to stand at 4° C. for 1 hour.

### Example 12

As in Examples 6 and 7, the composition of Example 12 was prepared with the components and amounts shown in Table 2 and used to fill a container. However, instead of a carboxyvinyl polymer, an (acrylates-acrylic acid alkyl (C10-30) crosspolymer (Pemulen EZ4U; Lubrizol) was used. The resulting emulsion was used to fill a container as in Examples 6 and 7 and allowed to stand at 4° C. for 1 hour.

### Comparative Example 2

Compositions were prepared in the same manner as in Examples 6 and 7, but without a carboxy vinyl polymer, used to fill a container as in Examples 6 and 7, and allowed to stand at 4° C. for 1 hour.

### Test Example 2. Stabilization Test

After returning to room temperature (n=3), the oil-based solid compositions for external use shown in Table 2 below were stored in a thermostatic chamber at 35° C. At this time, 300 ml of saturated brine (purified water with an excess amount of salt) in an open system was also placed in the thermostatic chamber to maintain high humidity conditions. The humidity was 70 to 75% RH. Each test sample was evaluated for separation of liquid components over time during storage. The liquid component was believed to be dipropylene glycol. As for the criteria for evaluating separation, droplets were observed under fluorescent light (Murakami Color Technology Laboratory, NL-500B, 10 amperes), and at least two out of three droplets were considered to be separated if the maximum diameter (long diameter) of the droplets was greater than 3 mm. The results are shown together in Table 2.
⊙: No separation after 3 days
◯: Separation observed after 3 days, but not before
Δ: Separation observed after 2 days, but not before
x: Separation observed after 1 day

### Table 2

The test results confirmed that separation was suppressed over time in the compositions of the examples.

### Test Example 3. Stabilization Test

The oil-based solid compositions for external use listed in Table 3 were prepared in the usual way. The components of the oil phase were prepared by mixing at 90° C. Separately, a pre-dispersed phase of carboxyvinyl polymer (Carbopol 980; Lubrizol) in squalane was prepared at room temperature. The pre-dispersed phase was then supplied to the oil phase at 90° C. Next, dipropylene glycol heated to 90° C. was added, and mixing performed in a homogenizer at 7,000 rpm for 1 minute at 90° C. Afterward, the composition was used to fill a jar-type container (Plastic Jar No. A-2, 11 cc: M.I. Chemical Co., Ltd.) and allowed to stand at 4° C. for 1 hour. After returning to room temperature (n=3), the oil-based solid compositions for external use were stored in a thermostatic chamber at 35° C. At this time, 300 ml of saturated brine (purified water with an excess amount of salt) in an open system was also placed in the thermostatic chamber to maintain high humidity conditions. The humidity was 70 to 75% RH. Each test sample was evaluated for separation of liquid components over time during storage. The liquid component was believed to be dipropylene glycol. The criteria for evaluating separation were the same as those in Test Example 2. The results are shown together in Table 3.

### Table 3

The test results confirmed that separation was suppressed in the compositions of the examples.

### Examples of Preparations

### Table 4

TABLE 1

| | | | C. Ex. 1 | Ex. 1 | Ex. 2 | Ex. 3 | Ex. 4 | Ex. 5 |
|---|---|---|---|---|---|---|---|---|
| Oil | Com- | Polyethylene | 7.5 | 7.5 | 7.5 | 7.5 | 7.5 | 7.5 |
| Phase | ponent | Paraffin wax | 5.0 | 5.0 | 5.0 | 5.0 | 5.0 | 5.0 |
| | (A) | Microcrystalline wax | 12.5 | 12.5 | 12.5 | 12.5 | 12.5 | 12.5 |
| | | Petroleum jelly | 2.5 | 2.5 | 2.5 | 2.5 | 2.5 | 2.5 |
| | | Cetyl alcohol | 5.0 | 5.0 | 5.0 | 5.0 | 5.0 | 5.0 |
| | | Jojoba oil | 15.5 | 15.2 | 15.2 | 14.5 | 13.5 | 12.5 |
| | | Squalane | 10.0 | 10.0 | 10.0 | 10.0 | — | — |
| | | Dipentaerythrityl tri- | 5.0 | 5.0 | 5.0 | 5.0 | 5.0 | 5.0 |
| | | polyhydroxystearate | | | | | | |
| | | Polyglyceryl-10 oleate | 5.0 | 5.0 | 5.0 | 5.0 | 5.0 | 5.0 |
| | | (VP/eicosene) | 0.5 | 0.5 | 0.5 | 0.5 | 0.5 | 0.5 |
| | | copolymer | | | | | | |
| | | PEG-30 di- | 0.5 | 0.5 | 0.5 | 0.5 | 0.5 | 0.5 |
| | | polyhydroxystearate | | | | | | |
| | | Carboxyvinyl polymer | — | — | 0.3 | 1.0 | — | — |

| | | | | | | | |
|---|---|---|---|---|---|---|---|
| Pre- | Squalane | — | — | | — | 10.0 | 10.0 |
| Dispersion | | | | | | | |
| Phase | Carboxyvinyl polymer | — | — | — | — | 2.0 | 3.0 |
| Polyhydric | Dipropylene glycol | 31.0 | 31.0 | 31.0 | 31.0 | 31.0 | 31.0 |
| Alcohol | | | | | | | |
| Phase | Carboxyvinyl polymer | — | 0.30 | — | — | — | — |

| | | | | | | |
|---|---|---|---|---|---|---|
| Total | 100 | 100 | 100 | 100 | 100 | 100 |
| Evaluation Results | X | ◯ | ◯ | ⊚ | ⊚ | ⊚ |

TABLE 2

| | | | C. Ex. 2 | Ex. 6 | Ex. 7 | Ex. 8 | Ex. 9 | Ex. 10 | Ex. 11 | Ex. 12 |
|---|---|---|---|---|---|---|---|---|---|---|
| Oil | Com- | Polyethylene | 7.0 | 7.0 | 7.0 | 7.0 | 7.0 | 7.0 | 7.0 | 7.0 |
| Phase | ponent | Paraffin wax | 9.0 | 9.0 | 9.0 | 9.0 | 9.0 | 9.0 | 9.0 | 9.0 |
| | (A) | Microcrystalline wax | 10.0 | 10.0 | 10.0 | 10.0 | 10.0 | 10.0 | 10.0 | 10.0 |
| | | Petroleum jelly | 2.5 | 2.5 | 2.5 | 2.5 | 2.5 | 2.5 | 2.5 | 2.5 |
| | | Cetanol | 5.0 | 5.0 | 5.0 | 5.0 | 5.0 | 5.0 | 5.0 | 5.0 |
| | | Tri(caprylic/capric) | 11.0 | 15.0 | 14.0 | 8.5 | 13.5 | 10.0 | 8.5 | 18.5 |
| | | glyceryl | | | | | | | | |
| | | Jojoba oil | 5.0 | — | — | 5.0 | 5.0 | 5.0 | 5.0 | — |
| | | Dipentaerythrityl tri- | 5.0 | 5.0 | 5.0 | 5.0 | — | 5.0 | 5.0 | — |
| | | polyhydroxystearate | | | | | | | | |
| | | Polyglyceryl-10 stearate | 5.0 | 5.0 | 5.0 | 5.0 | — | — | — | 5.0 |
| | | Polyglyceryl-10 oleate | — | — | — | — | 5.0 | 5.0 | 5.0 | — |
| | | PEG-30 di- | 0.5 | 1.0 | 1.0 | 1.0 | 1.0 | 0.5 | 0.5 | 1.0 |
| | | polyhydroxystearate | | | | | | | | |

| | | | | | | | | | |
|---|---|---|---|---|---|---|---|---|---|
| Pre- | Squalane | 10.0 | 10.0 | 10.0 | 10.0 | 10.0 | 10.0 | 10.0 | 10.0 |
| Dispersion | Carboxyvinyl polymer | — | 0.5 | 1.5 | 1.0 | 1.0 | — | — | — |
| Phase | Acrylates/(c10-30) alkyl | — | — | — | — | — | 1.00 | 1.50 | 2.00 |
| | acrylate crosspolymer | | | | | | | | |
| Polyhydric | Dipropylene glycol | 30.00 | 30.00 | 30.00 | 30.00 | 30.00 | 30.00 | 30.00 | 30.00 |
| Alcohol | | | | | | | | | |
| Phase | Hydrocortisone | — | — | — | 1.00 | 1.00 | — | 1.00 | — |

| | | | | | | | | |
|---|---|---|---|---|---|---|---|---|
| Total | 100.00 | 100.00 | 100.00 | 100.00 | 100.00 | 100.00 | 100.00 | 100.00 |
| Evaluation Results | X | ⊚ | ⊚ | ⊚ | ⊚ | ⊚ | ⊚ | ◯ |

TABLE 3

| | | | Ex. 13 | Ex. 14 | Ex. 15 | Ex. 16 |
|---|---|---|---|---|---|---|
| | | | % | % | % | % |
| | | Component Name | w/w | w/w | w/w | w/w |
| Oil | Com- | Polyethylene | 9.00 | 7.00 | 10.00 | 7.00 |
| Phase | ponent | Paraffin wax | 10.00 | 9.00 | 9.00 | 9.00 |
| | (A) | Microcrystalline | 15.00 | 10.00 | 13.00 | 10.00 |
| | | wax | | | | |
| | | Petroleum jelly | 2.50 | 2.50 | 2.50 | 2.50 |
| | | Cetanol | 6.00 | 5.00 | 6.00 | 5.00 |
| | | Tri(caprylic/ | 16.00 | 13.00 | 18.50 | 8.50 |
| | | capric)glyceryl | | | | |
| | | Jojoba oil | 7.00 | — | 5.00 | 5.00 |
| | | Dipentaerythrityl | 3.00 | 5.00 | 5.00 | 5.00 |
| | | tri- | | | | |
| | | poly- | | | | |
| | | hydroxystearate | | | | |
| | | Polyglyceryl-10 | 5.00 | 5.00 | 5.00 | 5.00 |
| | | oleate | | | | |
| | | PEG-30 di- | 0.50 | 0.50 | — | 0.50 |
| | | poly | | | | |

| | | | | | |
|---|---|---|---|---|---|
| | hydroxystearate | | | | |
| Pre- | Squalane | 10.00 | 10.00 | 10.00 | 10.00 |
| Dispersion | Carboxyvinyl | 1.00 | 3.00 | 1.00 | 2.50 |
| Phase | polymer | | | | |
| Polyhydric | Dipropylene | 15.00 | 30.00 | 15.00 | 30.00 |
| Alcohol | glycol | | | | |
| Phase | | | | | |

| | | | | |
|---|---|---|---|---|
| Total | 100.00 | 100.00 | 100.00 | 100.00 |
| Evaluation Results | ⊚ | ◯ | ⊚ | ⊚ |

TABLE 4

| | | | Prep. 1 | Prep. 2 | Prep. 3 | Prep. 4 | Prep. 5 | Prep. 6 | Prep. 7 | Prep. 8 |
|---|---|---|---|---|---|---|---|---|---|---|
| Oil | Com- | Polyethylene | 7 | 7 | 10 | 7 | 7 | 7 | 10 | 3 |
| Phase | ponent | Paraffin wax | 9 | 9 | 10 | 5 | 9 | 9 | 10 | — |
| | (A) | Microcrystalline wax | 5 | 10 | 10 | 10 | 10 | 10 | 10 | 3 |
| | | Sarashi beeswax | 1 | — | 3 | 5 | — | 5 | — | — |
| | | Carnauba wax | 1 | — | — | 3 | 3 | — | — | 1 |
| | | Cholesterol | 0.5 | — | — | — | 0.5 | 0.5 | — | — |
| | | Petroleum jelly | 2.5 | — | 2.5 | — | 2.5 | 3 | 3 | 2.5 |
| | | Cetanol | 5 | 5 | 5 | 5 | 5 | 5 | 5 | 5 |
| | | Menthol | 0.1 | — | — | — | 0.5 | — | 0.3 | — |
| | | Lidocaine | — | — | — | — | — | 1 | — | — |
| | | Tri(caprylic/ | 11.4 | 5 | 23.4 | 17.5 | 13.5 | 11.85 | 15 | 10 |
| | | capric)glyceryl | | | | | | | | |
| | | Jojoba oil | 5 | 4 | 5 | 10 | — | 10 | 6.55 | 12.5 |
| | | Isopropyl palmitate | — | — | 5 | 5 | — | 5 | 5 | — |
| | | Dipentaerythrityl tri- | 5 | 5 | 5 | 1 | 5 | 5 | 5 | — |
| | | polyhydroxystearate | | | | | | | | |
| | | Polyglyceryl-10 stearate | 5 | 5 | 5 | 5 | — | — | — | 5 |
| | | Polyglyceryl-10 oleate | — | — | — | — | 5 | 5 | 5 | — |
| | | PEG-30 di- | 0.5 | 1 | 1 | 0.5 | 1 | 0.5 | 1 | 1 |
| | | polyhydroxystearate | | | | | | | | |
| | | Dextrin palmitate | — | — | — | — | — | — | — | 5 |

| | | | | | | | | | |
|---|---|---|---|---|---|---|---|---|---|
| Pre- | Squalane | 10 | 10 | 10 | 10 | 6 | 10 | 10 | 10 |
| Dispersion | Liquid paraffin | — | 5 | — | — | — | 1 | 3 | 10 |
| Phase | Carboxyvinyl polymer | — | 3 | 0.1 | 1 | 1 | — | 1 | 1 |
| | Acrylates/(c10-30) alkyl | 1 | — | — | — | — | 1 | — | — |
| | acrylate crosspolymer | | | | | | | | |
| Polyhydric | Dipropylene glycol | 30 | 30 | 5 | — | 30 | 10 | 10 | 30 |
| Alcohol | Propylene glycol | — | — | — | 15 | — | — | — | — |
| Phase | Prednisolone | — | — | — | — | — | 0.15 | 0.15 | — |
| | valerate acetate | | | | | | | | |
| | Hydrocortisone | 1 | 1 | — | — | 1 | — | — | 1 |

| | | | | | | | | |
|---|---|---|---|---|---|---|---|---|
| Total (g) | 100 | 100 | 100 | 100 | 100 | 100 | 100 | 100 |

1. An oil-based solid composition for external use comprising:
(A) an oily component with a melting point of 55° C. or higher;
(B) a polyhydric alcohol; and
(C) an acrylic acid-based water-soluble polymer.

2. The oil-based solid composition for external use according to claim 1, wherein the composition is housed in a container with a cylindrical, polygonal, spherical, domed, hemispherical, conical, pyramidal, conical trapezoidal, or pyramidal trapezoidal shape.

3. The oil-based solid composition for external use according to claim 1, further comprising (D) a steroid.

4. The oil-based solid composition for external use according to claim 1, wherein (A) the oily component with a melting point of 55° C. or higher is one component or a combination of two or more components selected from the group consisting of paraffin wax, microcrystalline wax, ceresin, polyethylene, and beeswax.

5. The oil-based solid composition for external use according to claim 1, wherein the (B) polyhydric alcohol includes dipropylene glycol.

6. The oil-based solid composition for external use according to claim 1, wherein the (C) acrylic acid-based water-soluble polymer is one component or two or more components selected from the group consisting of polyacrylic acid, cross-linked polyacrylic acid, and alkyl acryl copolymers.

7. A method for increasing the stability of a composition comprising (A) an oily component with a melting point of 55° C. or higher and (B) a polyhydric alcohol, by the additional presence of (C) an acrylic acid-based water-soluble polymer in the composition.
